(19) United States
(12) Patent Application Publication
(10) Pub. No.: US 2017/0055947 A1
(43) Pub. Date: Mar. 2, 2017

(54) ULTRASOUND DIAGNOSTIC APPARATUS AND MEDIUM

(71) Applicant: Toshiba Medical Systems Corporation, Otawara-shi (JP)

(72) Inventors: Takashi OGAWA, Nasushiobara (JP); Yasushi Kamewada, Otawara (JP)

(73) Assignee: Toshiba Medical Systems Corporation, Otawara-shi (JP)

(21) Appl. No.: 15/244,472

(22) Filed: Aug. 23, 2016

(30) Foreign Application Priority Data
Aug. 25, 2015 (JP) 2015-165369

(51) Int. Cl. A61B 8/00; A61B 8/08; A61B 8/14

(52) U.S. Cl. CPC A61B 8/4461; A61B 8/145; A61B 8/46; A61B 8/5207; A61B 8/546; A61B 8/4405

(57) ABSTRACT

According to one embodiment, an ultrasound diagnostic apparatus includes a probe and processing circuitry. The probe includes an acoustic emission portion sealed in a liquid and a stirring mechanism which stirs the liquid. The processing circuitry is configured to determine a time to stir the liquid. The processing circuitry is configured to drive the stirring mechanism at the determined time to stir the liquid.

### CROSS-REFERENCE TO RELATED APPLICATIONS

This application is based upon and claims the benefit of priority from Japanese Patent Application No. 2015-165369, filed Aug. 25, 2015, the entire contents of which are incorporated herein by reference.

### FIELD

Embodiments described herein relate generally to an ultrasound diagnostic apparatus.

### BACKGROUND

Since an ultrasound diagnostic apparatus can visualize the internal body of an examinee in a non-invasive way and with no need to expose the patient to radiation, it is widely used for diagnosing a healthy person, a sick person, an injured person, an expecting person, etc. The ultrasound diagnostic apparatus is very small in comparison with an X-ray diagnostic apparatus, a CT apparatus, an MRI apparatus, etc., and can be easily moved to a bedside for the inspection of an examinee.

The ultrasound diagnostic apparatus comprises an ultrasound probe which is brought into contact with an examinee and transmits and receives ultrasonic waves, as well as a main apparatus including an image processor. In recent years, a personal computer installing dedicated software and a probe connected to the personal computer are known in the art as combination functioning as an ultrasound diagnostic apparatus. Also known in the art is an ultrasound diagnostic apparatus wherein the major functions are incorporated in a probe.

A probe includes a plurality of ultrasound transducers (acoustic emission portions) arranged as a one-dimensional array. When the transducers are driven in a predetermined pattern, ultrasonic beams are electronically scanned, and a two-dimensional image in a predetermined plane of the examinee can be obtained. In a recent ultrasound diagnostic apparatus, acoustic emission portions are mechanically swung (mechanical scan), and the internal body of the examinee is scanned spatially to collect three-dimensional biological information (volume data).

When the acoustic emission portions emit a large amount of ultrasound energy, heat is generated. If this state is left as it is, the temperature of the probe may become so high that the examinee may get burned. As a solution to this problem, the acoustic emission portions are sealed in a liquid (e.g., oil) having a large heat capacity, and the liquid is physically stirred to prevent the heat from staying locally. When the acoustic emission portions are swung, the liquid is stirred. As an alternative, the liquid may be stirred by a stirring mechanism provided independently.

The mechanical scan is useful not only in producing the three-dimensional volume data on the internal body of an examinee but also in preventing the probe temperature from becoming excessively high. In the existing technology, however, the above features of the mechanical scan are not positively used. That is, the liquid is stirred as a result of the three-dimensional scan, but not for any express purpose. In addition, in an examination protocol that does not require volume data, the liquid is not stirred at all. In order to prevent the temperature of the probe from increasing excessively, the diagnosis may have to be halted or the radiation energy of ultrasonic waves may have to be suppressed. As a result, the diagnostic efficiency may be degraded, and the resolution of an image may be lowered. Under the circumstances, there is a demand for technology for enabling efficient management of a probe temperature and preventing the diagnostic performance from deteriorating.

### BRIEF DESCRIPTION OF THE DRAWINGS

FIG. 1 is a perspective view illustrating an example of an ultrasound diagnostic apparatus according to an embodiment;

FIG. 2 is a functional block diagram showing an example of the main apparatus 100 and probe 10 shown in FIG. 1;

FIG. 3A shows an example of the probe 10;

FIG. 3B shows the example of the probe 10;

FIG. 4 is a flowchart illustrating an example of the processing performed by the ultrasound diagnostic apparatus of the embodiment;

FIG. 5 shows an example of a message displayed on a monitor 62 in step S8 shown in FIG. 4;

FIG. 6 is a schematic diagram illustrating an example of how a transducer unit 11 is swung in step S2 shown in FIG. 4;

FIG. 7 is a schematic diagram illustrating another example of how the transducer unit 11 is swung in step S2 shown in FIG. 4;

FIG. 8 is a schematic diagram illustrating how the swing angle and swing speed of the transducer unit 11 are related to the cooling effect;

FIG. 9 is a graph showing an example of an experimental result quantitatively illustrating the cooling effect produced by the swinging of the transducer unit 11;

FIG. 10A illustrates an example of how heat is distributed in a probe when the temperature is in the saturated state;

FIG. 10B illustrates an example of how heat is distributed in a probe when the temperature is in the saturated state;

FIG. 11A illustrates an example of how heat is distributed in a probe when the transducer unit 11 is swung from the state shown in FIG. 10A; and

FIG. 11B illustrates an example of how heat is distributed in a probe when the transducer unit 11 is swung from the state shown in FIG. 10B.

### DETAILED DESCRIPTION

In general, according to one embodiment, an ultrasound diagnostic apparatus includes a probe and processing circuitry. The probe includes an acoustic emission portion sealed in a liquid and a stirring mechanism which stirs the liquid. The processing circuitry is configured to determine a time to stir the liquid. The processing circuitry is configured to drive the stirring mechanism at the determined time to stir the liquid.

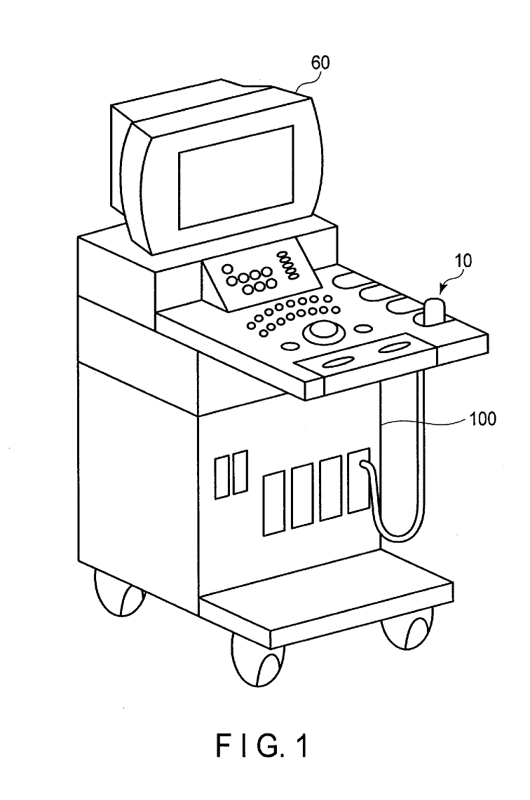

FIG. 1 is a perspective view illustrating an example of an ultrasound diagnostic apparatus according to an embodiment. The ultrasound diagnostic apparatus shown in FIG. 1 comprises a main apparatus 100, a probe (ultrasonic probe) 10 connected to the main apparatus 100, and a display 60. The main apparatus 100 is a so-called computer including a central processing unit (CPU) and a memory. A dedicated program is embedded in the main apparatus 100 (computer) or installed using a medium such as a CD-ROM, so as to achieve the functions of the ultrasound diagnostic apparatus.

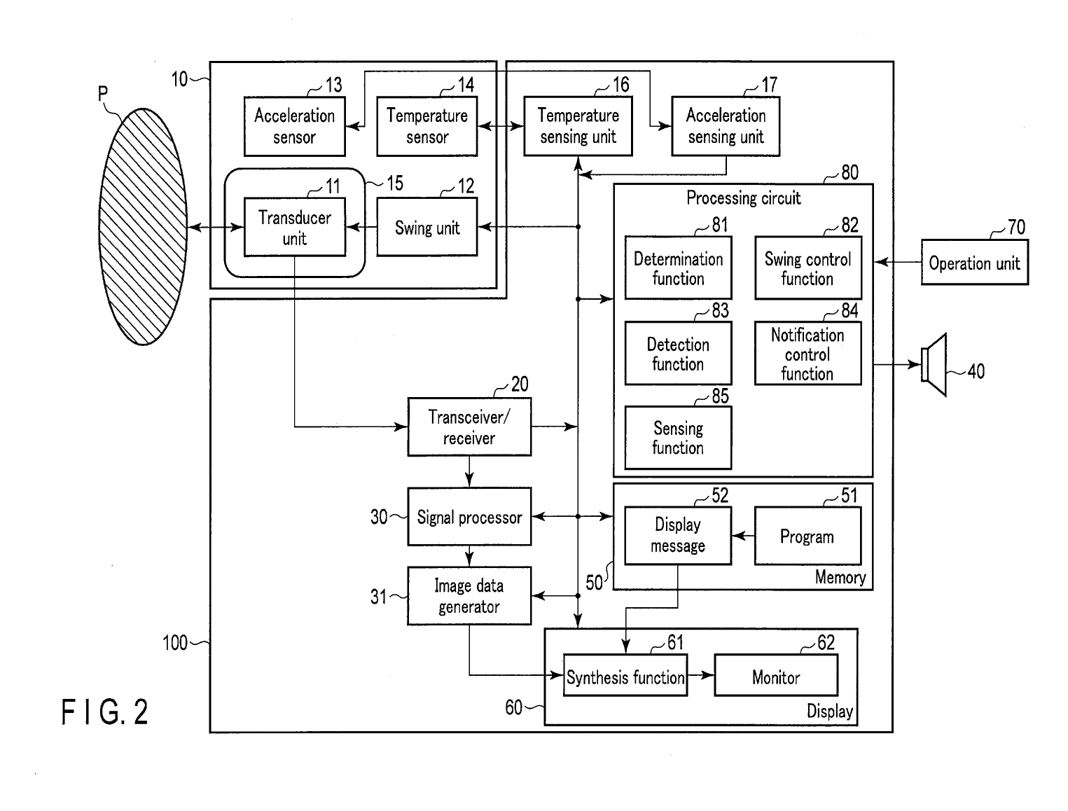

FIG. 2 is a functional block diagram showing an example of the main apparatus 100 and probe 10 shown in FIG. 1. The probe 10 includes a transducer unit 11, a swing unit 12, an acceleration sensor 13 and a temperature sensor 14. The transducer unit 11 is sealed in a hollow seal member 15 filled with a liquid. The liquid is preferably a substance having a large heat capacity (specific heat) such as oil.

The transducer unit 11 emits an ultrasonic wave to an examinee and receives an echo of the ultrasonic wave. The swing unit 12 is a mechanical scan mechanism for mechanically swinging the transducer unit 11. The acceleration sensor 13 detects the acceleration of the probe 10, namely, the movement of the probe 10. The temperature sensor 14 senses the temperature of the probe 10.

The main apparatus 100 includes a temperature sensing unit 16, an acceleration sensing unit 17, a transmitter/receiver 20, a signal processor 30, an image data generator 31, a speaker 40, a memory 50, a display 60, an operation unit 70 and a processing circuit 80. The temperature sensing unit 16 converts the temperature sensed by the temperature sensor 14 of the probe 10 into digital data, and supplies the digital data to the processing circuit 80. The acceleration sensing unit 17 converts the acceleration sensed by the acceleration sensor 13 of the probe 10 into digital data, and supplies the digital data to the processing circuit 80.

The transmitter/receiver 20 feeds the transducer unit 11 of the probe 10 to generate an ultrasonic wave. The ultrasonic wave is electronically scanned by the transmitter/receiver 20, thereby generating an ultrasonic beam that scans the internal body of the examinee P in a fan-like fashion. An ultrasonic echo reflected inside the examinee P returns to the transducer unit 11 and is received by the transmitter/receiver 20 as an electric signal.

The signal processor 30 performs various kinds of signal processing (including filtering processing, noise removal processing, and analog/digital conversion) for the electric signal output from the transmitter/receiver 20, so as to generate data such as B-mode data. The image data generator 31 generates ultrasonic image data based on the data generated by the signal processor 30. The ultrasonic image data is supplied to the synthesis function 61 of the display 60, and is displayed on the monitor 62, with various kinds of information superimposed thereon. The monitor 62 includes a CRT or a liquid crystal panel, and displays the ultrasonic image data output from the synthesis function 61.

The memory 50 stores various programs 51 and various display messages 52 to be displayed on the monitor 62. The operation unit 70 includes input devices (including various switches, a keyboard, a track ball and a mouse), a touch command screen, etc., and serves as a user interface that accepts a user operation. The user operates the operation unit 70 to enter imaging conditions, including the emission gain (output gain) of an ultrasonic wave, a dynamic range of the ultrasonic wave, a transmit frequency of the ultrasonic wave, a pulse recurrence frequency of the ultrasonic wave, a field-of-view depth, a viewing angle, a frame rate, a swing angle of the ultrasonic wave and a swinging range of the ultrasonic wave θr. The speaker 40 functions as a user interface as well and notifies the user of various information by generating sound and voice messages under the control of the processing circuit 80.

The processing circuit 80 has a determination function 81, a swing control function 82, a detection function 83, a notification control function 84 and a sensing function 85, which are processing functions of the embodiment.

The determination function 81 determines the time when the liquid in the seal member 15 is stirred. The time is determined for example as follows. When it is determined that the temperature of the probe 10 sensed by the temperature sensor 14 exceeds a predetermined value (e.g., 40° C.) or is expected to exceed the value, the liquid stirring time is a predetermined time (e.g., several milliseconds to several dozen seconds) after the time of determination.

The swing control function 82 drives the swing unit 12 at the times determined by the determination function 81 and swings the transducer unit 11 to stir the liquid. The transducer unit 11 assumes two states: an operating state where it is swung based on an examination protocol and a non-operating state. The swing control function 82 drives the swing unit 12 at the times determined by the determination function 81, irrespective of which examination protocol is selected. In other words, the swing control function 82 forcibly swings the transducer unit 11.

The notification control function 84 notifies the user (a doctor or a technician) that the swing unit 12 is being driven, by issuing a display message or a voice message. The sensing function 85 analyzes features of the ultrasonic image data generated by the image data generator 31, and determines based on the analysis result whether or not the probe 10 is left exposed to the air.

The detection function 83 detects a so-called idle time generated in the diagnosis employing the ultrasound diagnostic apparatus. In other words, the detection function 83 detects a time interval within a series of operations. The determination function 81 determines that the time interval detected by the detection function 83 is a time when the liquid in the seal member should be stirred.

The time interval may be a predetermined time immediately after the ultrasound diagnostic apparatus is turned on. Alternatively, the time interval may be a predetermined time after the acceleration sensor 13 continues to detect the non-operating state of the probe 10 for more than a predetermined length of time (e.g., several dozen seconds). Alternatively, the time interval may be a predetermined time after the operation unit 70 is not operated by the user for more than a predetermined length of time.

Alternatively, the time interval may be a predetermined time after the transition to a freeze state is designated by the operation unit 70. Alternatively, the time interval may be a predetermined time after the sensing function 85 detects that the probe 10 is left exposed to the air for more than a predetermined length of time.

The determination function 81, the swing control function 82, the detection function 83, the notification control function 84 and the sensing function 85 are executable by a computer, and a program 51 for this is stored in the memory 50. The processing circuit 80 is a processor which realizes the functions corresponding to the routines of the program 51 by reading the program 51 from the memory 50 and executing the program 51. In other words, the processing circuit 80 has the functions shown in FIG. 2 when it reads the program 51.

In FIG. 2, the determination function 81, the swing control function 82, the detection function 83, the notification control function 84 and the sensing function 85 are shown as being attained by the same processing circuit 80. This is not restrictive, and the determination function 81, the swing control function 82, the detection function 83, the notification control function 84 and the sensing function 85 may be attained by a combination of a number of processors.

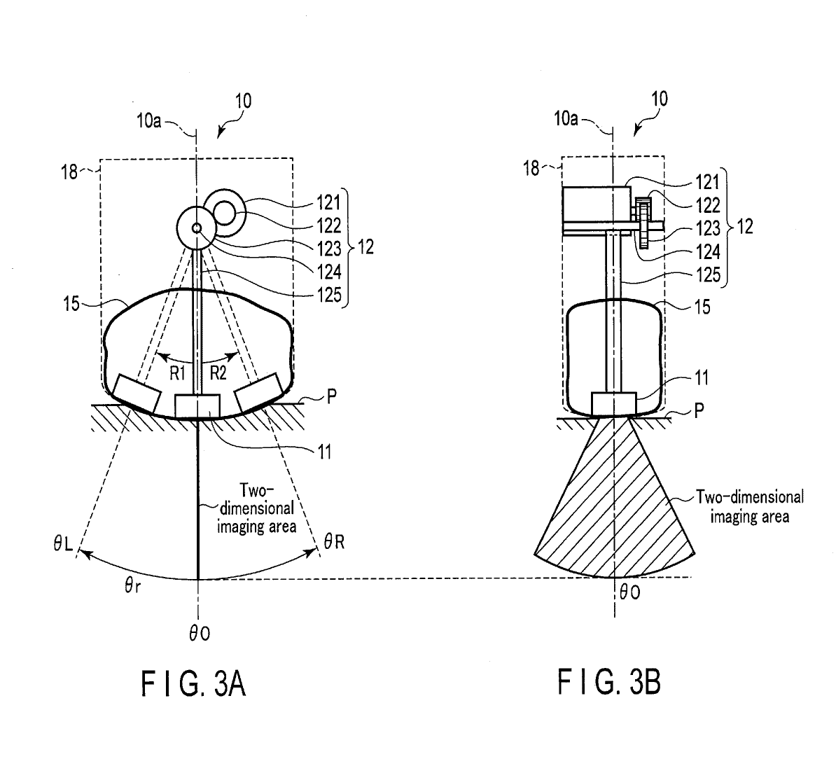

FIGS. 3A and 3B show an example of the probe 10. FIG. 3A illustrates a direction in which the transducer unit 11 of the probe 10 swings. FIG. 3B illustrates how the probe 10 depicted in FIG. 3A looks like when it is viewed from a position which is rotated 90° around the central axis 10a. The seal member 15 and the swing unit 12 are contained in a case 18 and protected from impact.

Referring to FIG. 3A, the swing unit 12 comprises: a motor 121 serving as a driving force generator that generates a driving force for swinging the transducer unit 11; a first gear 122 fixed to the rotating shaft of the motor 121; a second gear in engagement with the first gear 122; a swing shaft 124 extending through the center of rotation of the second gear 123 and fixed to the second gear 123; and an arm connected to the swing shift at one end and holding the transducer unit 11 at the other end. When the arm 125 is on the central axis 10a of the probe 10, the swing angle of the transducer unit 11 is referred to as reference angle θ0.

When the arm is moved back and forth by the rotation of the motor 121, the swing unit 12 swings the transducer unit 11 in an arrow R1 direction and in an arrow R2 direction opposite to the arrow R1 direction. The transducer unit 11 describes an arc wherein the center is the swing shaft 124 and the radius is the length of the arm 125. The swingable range θr of the transducer unit 11 is between swing angle θL which is away from the reference angel θ0 in the arrow R1 direction and swing angle θR which is away from the reference angel θ0 in the arrow R2 direction.

The transducer unit 11 includes a plurality of transducers (N transducers) arranged in a direction perpendicular to the swinging direction. Supplied with a driving force, the transducers generate ultrasonic waves. When the transducer unit 11 is stationary, the two-dimensional imaging area (indicated by the oblique hatching) in the examinee P is electronically scanned, as shown in FIG. 3B. When the transducer unit 11 swings, the two-dimensional imaging area is sequentially scanned (mechanical scan), and three-dimensional volume data can be obtained thereby. This processing may be referred to as a 4D scan, with the time added as one dimension. A description will now be given of an operation of the apparatus having the above structure.

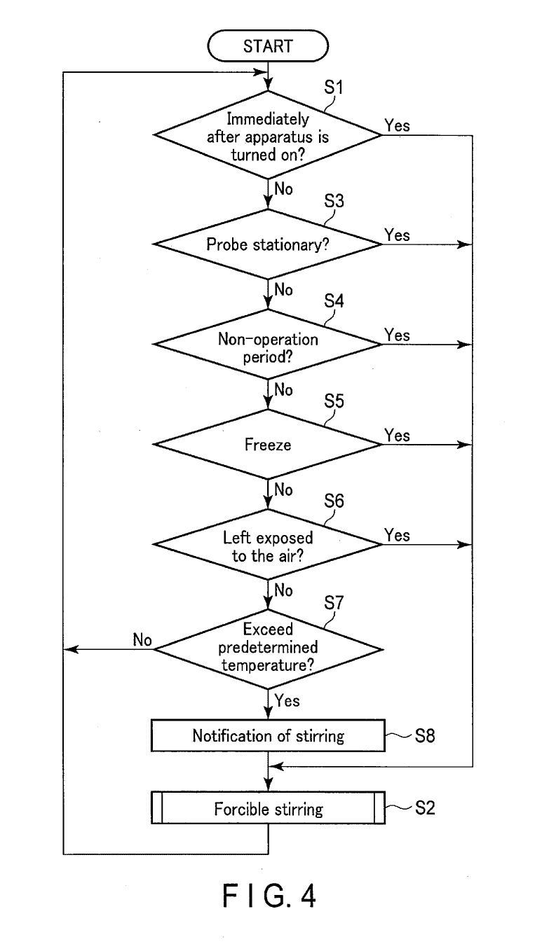

FIG. 4 is a flowchart illustrating an example of the processing performed by the ultrasound diagnostic apparatus of the embodiment. When the processing starts, the processing circuit 80 of the ultrasound diagnostic apparatus determines whether the present time is immediately after the ultrasound diagnostic apparatus is turned on (step S1). If YES in step S1, the determination function 81 determines that the swing unit 12 should be driven then, and the swing control function 82 drives the swing unit 12 and forcibly swings the transducer unit 11 (step S2).

If the time is not immediately after the ultrasound diagnostic apparatus is turned on (No in step S1), the processing circuit 80 determines whether or not the probe 10 is stationary (step S3). If Yes in step S3, the determination function 81 assumes that an idle time is generated and determines that the swing unit 12 should be driven. In accordance therewith, the swing control function 82 forcibly swings the transducer unit 11 (step S2).

If the probe 10 is moving (No in step S3), the processing circuit 80 determines whether there is a non-operation period during which the operation unit 70 is not operated (step S4). If such a period exists and is longer than a predetermined period, then the processing circuit 80 determines Yes in step S4. In this case, the swing control function 82 forcibly swings the transducer unit 11 (step S2).

If No in step S3, the processing circuit 80 determines whether a freeze operation is performed (step S5). If the freeze button is operated (Yes in step S5), the determination function 81 notifies the swing control function 82 of the generation of a time interval, and the swing control function 82 forcibly swings the transducer unit 11 (step S2).

If the freeze operation is not performed (No in step S5), the processing circuit 80 determines whether the probe 10 is left exposed to the air (step S6). If it is determined that the probe 10 is left exposed to the air (Yes in step S6), the swing control function 82 forcibly swings the transducer unit 11.

If No in step S6, the processing circuit 80 determines whether the temperature of the probe 10 exceeds a predetermined value (step S7). If the temperature exceeds the predetermined value (Yes in step S7), the notification control function 84 causes the monitor 6 to display a message indicating that the transducer unit 11 is swinging, namely, the liquid is being stirred (step S8), and the swing control function 82 forcibly swings the transducer unit 11.

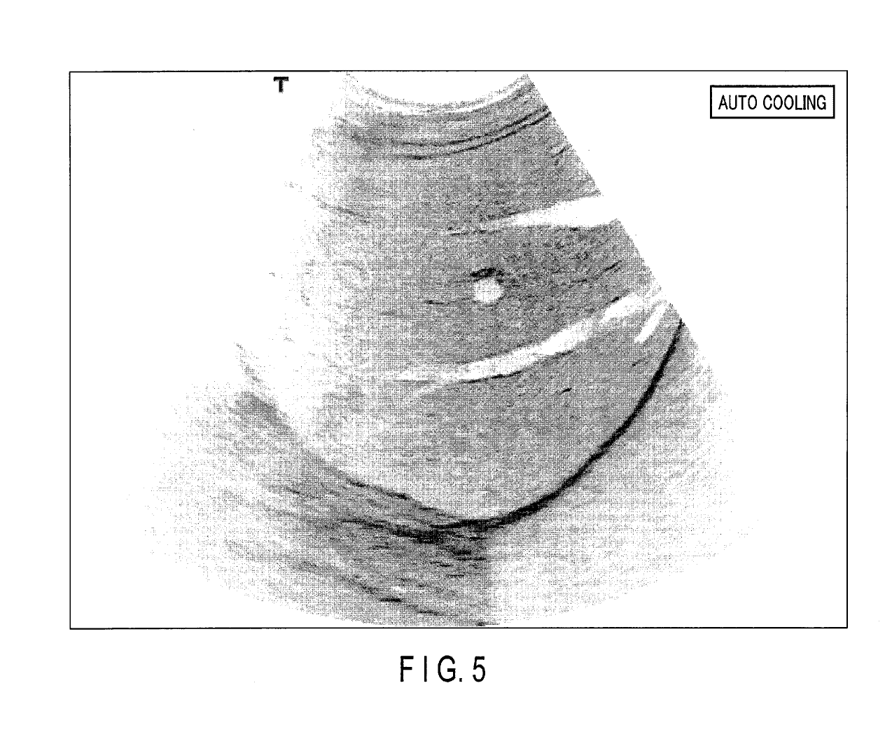

FIG. 5 shows an example of the message displayed on the monitor 62 in step S8 shown in FIG. 4. The reason why the transducer unit 11 is swung at a time other than an idle time is that such a swinging motion has to be performed due to the excessive temperature rise of the probe 10. In this case, the message shown in FIG. 5 (“AUTO COOLING”) is displayed together with an ultrasonic image, so as to notify the user that the transducer unit 11 is being driven (it is being cooled).

After the transducer unit 11 is forcibly swung in step S2, the processing flow returns to step S1, and the same processing is repeated again. If the determination made in step S1 and S3-S8 indicates No, the processing flow returns to step S1, and the same processing is repeated again.

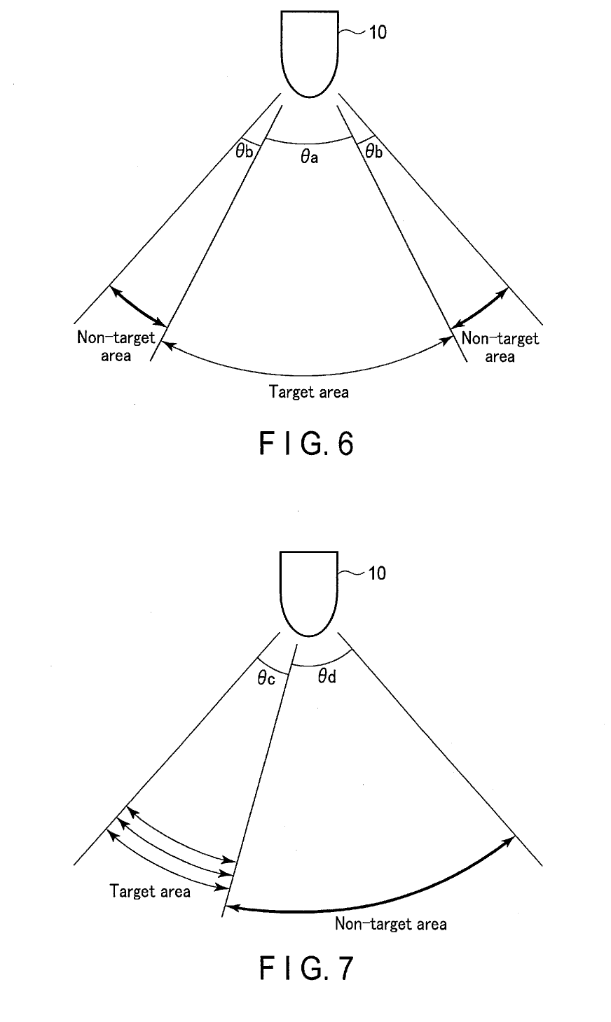

FIGS. 6 and 7 are schematic diagrams illustrating examples of how the transducer unit 11 is swung in step S2 shown in FIG. 4. Referring to FIG. 6, if a time interval is generated when the area of angle θa including the central axis of the probe 10 is scanned, then the swinging control unit 82 swings the transducer unit 11 such that the transducer unit 11 is directed toward the areas of angle θb (non-target areas), which are outward of a target area. In order to achieve efficient cooling effect, the transducer unit 11 should be swung desirably at a higher speed when it is directed toward the non-target areas than when it is directed toward the target area. More desirably, the transducer unit 11 should be swung at the highest speed when it is directed toward the non-target areas.

The swing speed is an index corresponding to the number of mechanical scans performed per unit time. If a scan is performed five times within one second in the normal examination mode, the swing speed should be twice as high in the swinging mode, namely, ten times within one second. It can be readily appreciated that the cooling effect can be improved, accordingly.

When conditions permit, a mechanical scan may be performed at a speed higher than that of normal examination mode in all areas indicated by (θb+θa+θb). By performing the mechanical scan in this manner, the cooling effect can be maximal. It is also effective to increase the number of times the swinging operation is performed. A sufficient cooling effect can be obtained by making either the swing angle or the swing speed maximal. In other words, the probe cooling effect not obtained in the normal examination mode can be attained by setting the swing angle and swing speed at values larger than those set in the normal examination mode.

As shown in FIG. 7, when the area of angle θc is scanned as a target area, the non-target area (θd) may be scanned only once each time the target area is scanned ten times. In this case as well, the probe cooling effect can be obtained. The number of times the non-target area is scanned is not limited to this example, and the cooling effect increases in accordance with an increase in the scan speed of the non-target area (θd).

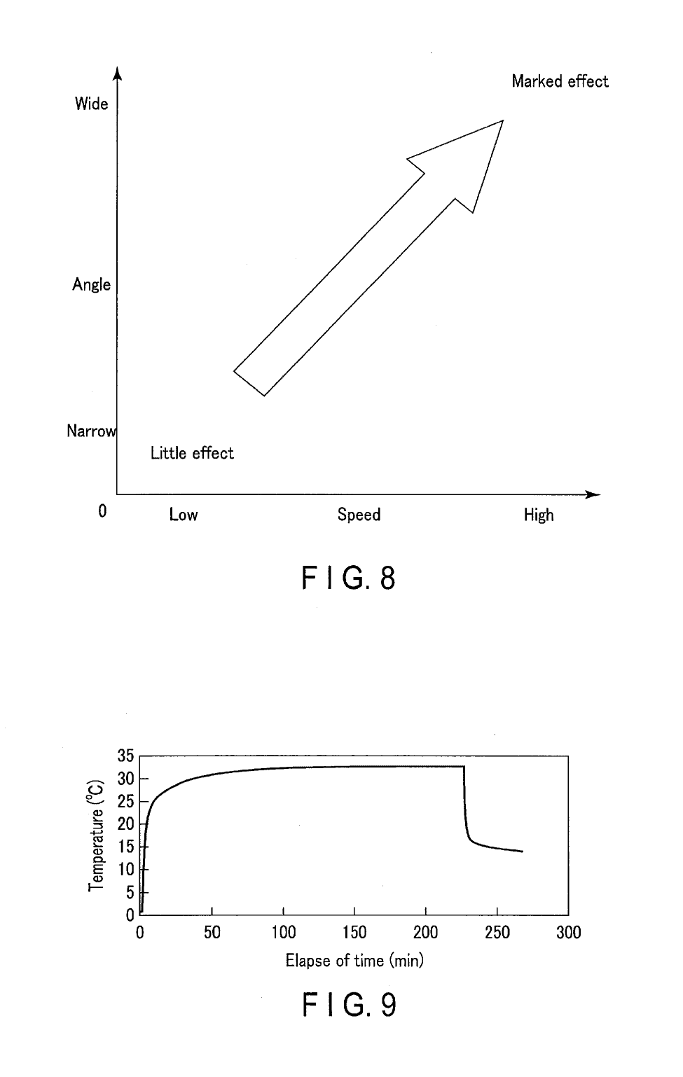

FIG. 8 is a schematic diagram illustrating how the swing angle and swing speed of the transducer unit 11 are related to the cooling effect. If the swing angle is narrow and the swing speed is low, the cooling effect is minimal. The cooing effect is improved if the swing angle is widened and/or the swing speed is increased. The cooling effect can be improved to a certain extent if the swing angle is widened or if the swing speed is increased.

FIG. 9 is a graph showing an example of an experimental result quantitatively illustrating the cooling effect produced by the swinging of the transducer unit 11. When the transducer unit 11 is fed and an ultrasonic wave is generated, the temperature increases rapidly and is saturated. If the transducer unit 11 is swung in this saturated state, the temperature falls by about 15° C. in no time at all, as can be seen in the graph.

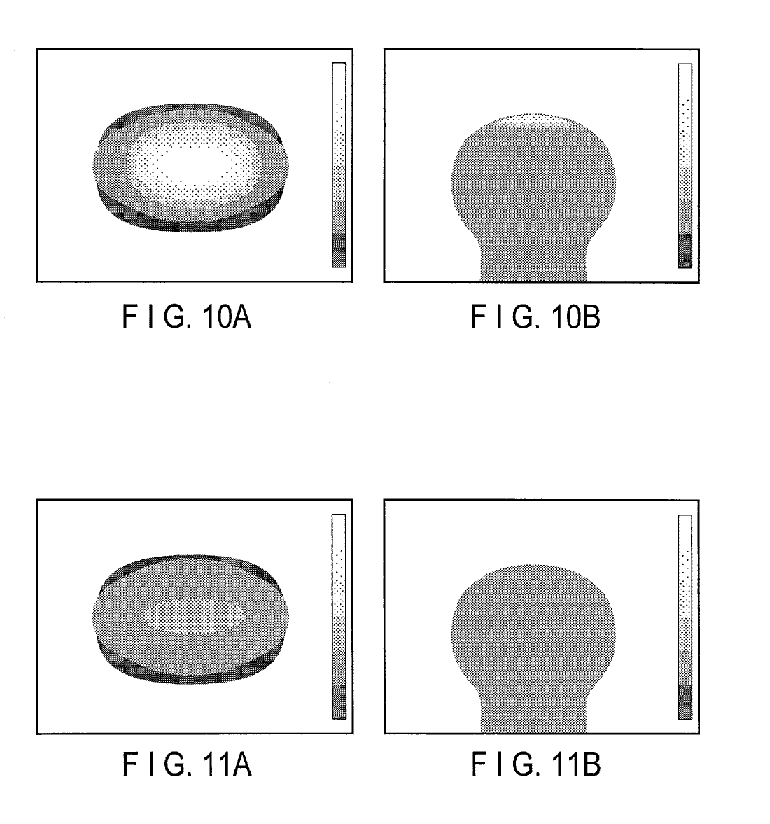

FIGS. 10A and 10B illustrate an example of how heat is distributed in a probe when the temperature is in the saturated state. As can be seen in the top view shown in FIG. 10A and the side view shown in FIG. 10B, the temperature of the probe is high particularly at the tip end (a high temperature portion is indicated as a white portion). If the transducer unit 11 is swung in this saturation state, the temperature distribution becomes substantially uniform, as shown in FIGS. 11A and 11B.

In the embodiment described above, the detection function 83 detects a time interval (idle time) generated in the sequence of ultrasonic diagnosis, and the transducer unit 11 is swung during such a time interval. In actual examination, the transducer unit 11 may be swinging at all times. In this case as well, the transducer unit 11 may be swung for the express purpose of cooling, as long as the swinging does not have adverse effects on the examination being carried out. The probe 10 can maintain a uniform temperature distribution by automatically swinging the transducer unit 11 in such a manner as not to cause adverse effects on the examination being carried out.

Since the temperature of the probe can be effectively managed, the radiation energy of ultrasonic waves does not have to be controlled. As a result, an ultrasound diagnostic apparatus and a program can be provided which enable examination to be carried out with high sensitivity and ensure reliable diagnostic performance.

According to the embodiment, the temperature of the probe can be effectively managed, and the diagnostic performance of the ultrasound diagnostic apparatus can be enhanced.

If, for some reason or other, the swinging operation has to be performed irregularly, the user is notified of this state by a message displayed on the monitor 62. As a result, even if an image is blurred by mismatching of frame rates, the user can readily understand why the image blurring occurs.

The above-described embodiment is not restrictive and can be modified in various manners in practice without departing from the gist of the embodiment. Examples of specific modifications include, for example, the following:

(1) In the above embodiment, the liquid is stirred by swinging the transducer unit 11. In place of this, a stirring mechanism may be provided independently of the transducer unit 11, and the liquid may be stirred by this stirring mechanism. The stirring mechanism may be an electrically-vibrating stirrer. Also, a non-contact stirring mechanism may be employed. To be specific, a magnetized stirrer may be contained in the seal member 15, and the stirrer is permitted to move in the seal member 15 in response to variations in the electromagnetic field, thereby stirring the liquid.

(2) The time to swing the transducer unit 11 is not necessarily determined by the conditions shown in FIG. 4. It may be a very short time when the transmit/receive frequency of ultrasonic waves is switched, when the diagnostic mode is switched or when the setting conditions are changed, as long as the swinging of the transducer unit 11 does not have adverse effects on the diagnosis. The time to swing the transducer unit 11 may be a time when the user enters various data to the apparatus and a time when the operator and the examinee talk to each other.

(3) The method in which the user is notified of the swinging state of the transducer unit 11 is not limited to the message shown in FIG. 5. For example, a light emitting diode (LED) provided for the operation unit 70 or for the probe 10 may be lit. Alternatively, the speaker 40 may output a voice message or beep sound. Furthermore, the probe 10 may be physically vibrated.

(4) In the above embodiment, the transducer unit 11 is swung and the temperature distribution in the probe 10 is made uniform, whereby the need to control the radiation energy of ultrasonic waves is eliminated. If, for some reason or other, the temperature distribution cannot be made sufficiently uniform, the radiation energy may be automatically suppressed. The basic premise for this is that the examinee is not exposed to an excessive amount of heat.

(5) The temperature sensing unit 16 and the acceleration sensing unit 17 shown in FIG. 2 are not indispensable. The speaker 40 is not indispensable, either.

The functions described in connection with the above embodiment may be attained by installing a program for executing the processing in a computer and developing the program on a memory. The program for permitting the computer to execute the processing can be stored in a computer-readable storage medium, such as a magnetic disk (a floppy disk, a hard disk, or the like), an optical disk (a CD-ROM, a DVD, or the like), and a semiconductor memory, and can be distributed.

The term “processor” described in the above can be realized, for example, by: a central processing unit (CPU) and a graphics processing unit (GPU); an application specific integrated circuit (ASIC), a simple programmable logic device (SPLD) and a complex programmable logic device (CPLD); a field programmable gate array (FPGA); or the like. The processor reads the programs stored in the memory 50 and executes them to realize the respective functions.

The operation programs may be incorporated or embedded in the circuits of respective processors, instead of storing them in the memory 50 of the processing circuit 80. In this case, the processors read the programs incorporated in their circuits and execute the programs to realize the respective functions.

While certain embodiments have been described, these embodiments have been presented by way of example only, and are not intended to limit the scope. Indeed, the novel embodiments described herein may be embodied in a variety of other forms; furthermore, various omissions, substitutions and changes in the form of the embodiments described herein may be made without departing from the spirit. The accompanying claims and their equivalents are intended to cover such forms or modifications as would fall within the scope and spirit.

1. An ultrasound diagnostic apparatus comprising:
a probe including an acoustic emission portion sealed in a liquid and a stirring mechanism which stirs the liquid; and
processing circuitry configured to:
determine a time to stir the liquid, and
drive the stirring mechanism at the determined time to stir the liquid.

2. The apparatus of claim 1, wherein the processing circuitry is further configured to:
detect a time interval during diagnosis, and
determine the detected time interval as the time to stir the liquid.

3. The apparatus of claim 2, wherein the processing circuitry is further configured to detect, as the time interval, a predetermined time which is immediately after the apparatus is turned on.

4. The apparatus according to claim 2, further comprising:
a motion sensor configured to detect a motion of the probe,
wherein the processing circuitry is further configured to detect, as the time interval, a predetermined time which is after a given length of time during which a stationary state of the probe is kept sensed by the motion sensor.

5. The apparatus according to claim 2, further comprising:
a user interface configured to accept a user operation,
wherein the processing circuitry is further configured to detect, as the time interval, a predetermined time which is after a given length of time during which no user operation is entered to the user interface.

6. The apparatus according to claim 2, further comprising:
a user interface configured to accept a user operation,
wherein the processing circuitry is further configured to detect, as the time interval, a predetermined time which is after an instruction for switching the apparatus to a freeze state is entered from the user interface.

7. The apparatus according to claim 2, further comprising:
an image generator configured to process acoustic data obtained by the probe and generate an ultrasonic image,
wherein the processing circuitry is further configured to:
detect whether the probe is left exposed to the air based on a feature of the ultrasonic image, and
detect, as the time interval, a predetermined time which is after a given length of time during which the probe is left exposed to the air.

8. The apparatus according to claim 1, further comprising:
a temperature sensor configured to detect a temperature of the probe,
wherein the processing circuitry is further configured to determine the time to stir the liquid, based on the detected temperature of the probe.

9. The apparatus according to claim 1, further comprising:
notification means for notifying a user that the stirring mechanism is being driven.

10. The apparatus of claim 1, wherein the stirring mechanism is a mechanical scan mechanism which mechanically swings the acoustic emission portion.

11. The apparatus of claim 10, wherein the processing circuitry is further configured to maximize at least one of a swing angle and a swing speed of the acoustic emission portion when the liquid is stirred.

12. The apparatus of claim 10, wherein the processing circuitry is further configured to swing the acoustic emission portion at such an angle as enables an area different from a diagnosis target area to be scanned.

13. The apparatus of claim 10, wherein the processing circuitry is further configured to swing the acoustic emission portion at a higher speed in an angular range which enables an area different from a diagnosis target area to be scanned than in an angular range which enables the diagnosis target area to be scanned.

14. The apparatus of claim 1, wherein the stirring mechanism is a non-contact stirring mechanism wherein a magnetized stirrer is movable in response to variations in an electromagnetic field in a non-contact manner.

15. A non-transitory computer-readable medium storing a program executed by a computer, the program comprising:
determining a time to stir a liquid of a probe, the probe including an acoustic emission portion sealed in the liquid and a stirring mechanism which stirs the liquid; and
driving the stirring mechanism at the determined time to stir the liquid.

16. The medium of claim 15, wherein the program further comprising:
detecting a time interval during diagnosis, and
the determining comprises determining the detected time interval as the time.

17. The medium of claim 16, wherein the program further comprising:
processing acoustic data obtained by the probe and generating an ultrasonic image,
detecting whether the probe is left exposed to the air based on a feature of the ultrasonic image, and
the detecting comprising detecting, as the time interval, a predetermined time which is after a given length of time during which the probe is left exposed to the air.
